(19) United States
(12) Patent Application Publication
(10) Pub. No.: US 2024/0016634 A1
(43) Pub. Date: Jan. 18, 2024

(54) AUXETIC DEVICE DELIVERY APPARATUS AND METHOD

(71) Applicant: OREGON HEALTH & SCIENCE UNIVERSITY, Portland, OR (US)

(72) Inventors: Ramsey Al-Hakim, Portland, OR (US); Khashayar Farsad, Portland, OR (US); John A. Kaufman, Portland, OR (US)

(73) Assignee: OREGON HEALTH & SCIENCE UNIVERSITY, Portland, OR (US)

(21) Appl. No.: 18/025,489

(22) PCT Filed: Oct. 13, 2021
(86) PCT No.: PCT/US2021/054722

§ 371 Date: Mar. 9, 2023

Related U.S. Application Data
(60) Provisional application No. 63/090,847, filed on Oct. 13, 2020.

(51) Int. Cl. A61F 2/966; A61F 2/95

(52) U.S. Cl. CPC A61F 2/966; A61F 2/9517; A61F 2250/0098

(57) ABSTRACT

Embodiments include a delivery device for an auxetic device. The delivery device is comprised of an outer constraining device and a longitudinal controlling device disposed concentrically within the outer constraining device and offset to form a chamber for receiving the auxetic device. A mechanism coupled to the outer constraining device and the longitudinal controlling device allows the chamber size to adjust longitudinally as the auxetic device is deployed. Other embodiments may be described and/or claimed.

This application claims the benefit of United States Provisional Patent Applications No. 63/090,847, filed Oct. 13, 2020 entitled AUXETIC DEVICE DELIVERY APPARATUS AND METHOD, the entire disclosure of which is hereby incorporated by reference.

### TECHNICAL FIELD

Disclosed embodiments are directed to the delivery of medical devices and in particular, to apparatuses and methods for delivery and deployment of an auxetic device, such as a stent.

### BACKGROUND

Auxetic stents have a unique benefit in application to the veins, and potentially other tubular structures in the body with similar biomechanical properties such as biliary ducts, genitrourinary tract, tracheobronchial tree, gastrointestinal tract, lymphatic channels, salivary ducts, eustachian tubes, arteries, and other tubular structures in the body. Auxetic stents, and stents in general, may be inserted into a patient using minimally invasive techniques, such as via a catheter, to avoid the trauma imposed by general surgery.

The background description provided herein is for the purpose of generally presenting the context of the disclosure. Unless otherwise indicated herein, the materials described in this section are not prior art to the claims in this application and are not admitted to be prior art by inclusion in this section.

### BRIEF DESCRIPTION OF THE DRAWINGS

Embodiments will be readily understood by the following detailed description in conjunction with the accompanying drawings. To facilitate this description, like reference numerals designate like structural elements. Embodiments are illustrated byway of example, and not by way of limitation, in the figures of the accompanying drawings.

FIG. 1 is a pair of radiograms illustrating an example auxetic device delivery apparatus before delivery of the device, and after delivery and expansion of the auxetic device, according to various embodiments.

FIG. 2 is a diagram of an example auxetic device delivery apparatus illustrating the various components of the apparatus, according to various embodiments.

FIG. 3 is a flowchart of operations for an example method of deploying an auxetic device with the example delivery apparatus of FIG. 2, according to various embodiments.

FIG. 4 is a cutaway view of an example control mechanism for an auxetic device delivery apparatus, such as the example apparatus of FIG. 2, according to various embodiments.

FIG. 5A depicts a first example placement of an auxetic stent within an auxetic device delivery apparatus with a pusher positioned at least partially within the stent, according to various embodiments.

FIG. 5B depicts a second example placement of an auxetic stent within an auxetic device delivery apparatus with the pusher abutting the end of the stent, according to various embodiments.

### DETAILED DESCRIPTION

In the following detailed description, reference is made to the accompanying drawings which form a part hereof wherein like numerals designate like parts throughout, and in which is shown by way of illustration embodiments that may be practiced. It is to be understood that other embodiments may be utilized and structural or logical changes may be made without departing from the scope of the present disclosure. Therefore, the following detailed description is not to be taken in a limiting sense, and the scope of embodiments is defined by the appended claims and their equivalents.

Aspects of the disclosure are disclosed in the accompanying description. Alternate embodiments of the present disclosure and their equivalents may be devised without parting from the spirit or scope of the present disclosure. It should be noted that like elements disclosed below are indicated by like reference numbers in the drawings.

Various operations may be described as multiple discrete actions or operations in turn, in a manner that is most helpful in understanding the claimed subject matter. However, the order of description should not be construed as to imply that these operations are necessarily order dependent. In particular, these operations may not be performed in the order of presentation. Operations described may be performed in a different order than the described embodiment. Various additional operations may be performed and/or described operations may be omitted in additional embodiments.

For the purposes of the present disclosure, the phrase “A and/or B” means (A), (B), or (A and B). For the purposes of the present disclosure, the phrase “A, B, and/or C” means (A), (B), (C), (A and B), (A and C), (B and C), or (A, B and C).

The description may use the phrases “in an embodiment,” or “in embodiments,” which may each refer to one or more of the same or different embodiments. Furthermore, the terms “comprising,” “including,” “having,” and the like, as used with respect to embodiments of the present disclosure, are synonymous.

Based on testing of auxetic stents, it is recognized that a unique delivery device is desirable for auxetic stents. Currently known deployment devices for stents may foreshorten or stay the same length from loading to deployment. However, there are no deployment devices specifically designed for delivery of auxetic devices, including auxetic stents. Because currently known deployment devices either foreshorten or stay a constant length, such devices are not ideal for auxetic devices, which elongate as they are expanded. Consequently, such deployment devices may not sufficiently contain an auxetic device as it lengthens and expands, rendering placement and achieving a desired expansion/shape problematic. In some scenarios, a deployment device may resist the device as it lengthens, potentially resulting in undesirable device deformation. Such deficiencies may unnecessarily complicate the deployment of an auxetic device.

Embodiments of disclosed delivery devices, for devices with auxetic properties, can accommodate the active elongation of an auxetic device during deployment. This may include active or passive elongation of the auxetic device during deployment, in either a linear or a non-linear fashion. The delivery device may be able to keep the auxetic device properly contained through the delivery process, preventing the auxetic device from unnecessarily interacting with surrounding structures during positioning, by employing delivery components that can move at different speeds relative to each other. The delivery device, by accommodating the longitudinal lengthening of the auxetic device via a pushing component that moves at a first speed and a sheath that moves at a second speed, can keep the auxetic device properly constrained as it lengthens to facilitate delivery to the correct location, and/or avoid imparting any resistance to the lengthening or expansion so as to avoid any unintended and/or undesirable deformation. The delivery device can be utilized to deliver auxetic devices/stents in tubular structures in the body including, but not limited to, biliary ducts, genitourinary tract, tracheobronchial tree, gastrointestinal tract, lymphatic channels, salivary ducts, eustachian tubes, arteries, and other tubular structures in the body.

In some embodiments, the axial/longitudinal length of the auxetic device can be either actively or passively controlled during deployment, such as by a longitudinal controlling device or mechanism. The axial/longitudinal length control can be configured for linear or non-linear elongation during deployment. Example longitudinal controlling mechanisms or devices can be active (such as a longitudinal controlling device with a gear mechanism control) or passive (such as a longitudinal controlling device with a spring mechanism control).

In an active control embodiment, an outer constraining device, such as a catheter that contains a stent or other auxetic device for delivery and placement, is coupled to the longitudinal controlling device via a gear mechanism. The outer constraining device/catheter may also be coupled to a user-controlled mechanism for retraction (such as a thumbwheel). During retraction of the outer constraining device/catheter by the user-controlled mechanism, the coupled gear mechanism between the outer constraining device/catheter and longitudinal controlling device enables simultaneous retraction of the longitudinal controlling device at a ratio determined by the gear ratio(s) of the gear mechanism which may be comprised of multiple gears. This will produce simultaneous retraction of both the outer constraining device/catheter and the longitudinal controlling device, potentially at different rates, with only a single input from the user. This can control elongation of the auxetic device/stent from the constrained length to unconstrained length (nominal length) during deployment.

Embodiments of the disclosed delivery device allow active or passive control of the axial/longitudinal aspect of devices during delivery. This may be particularly applicable for delivery/deployment of auxetic devices which elongate during deployment, such as an auxetic stent. By controlling the axial/longitudinal length of the auxetic device during deployment, the mechanical forces of the auxetic device can be optimally applied to the recipient structure, such as a vessel in the body or any other tubular structure in the body.

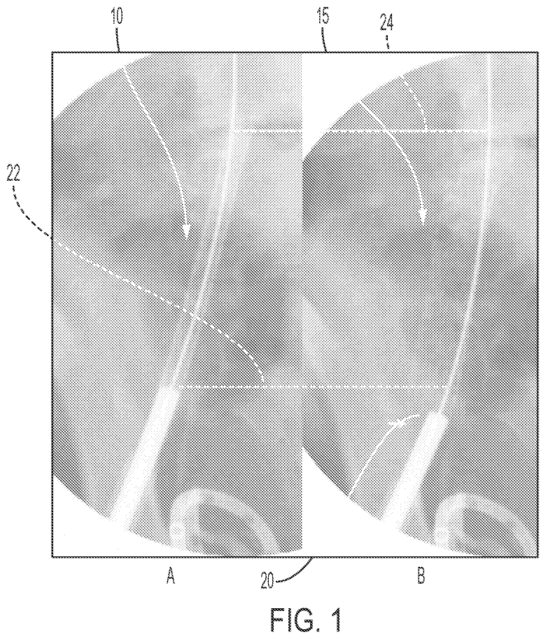

FIG. 1 is a radiogram illustrating deployment of an auxetic stent 10, and how radial expansion of the stent 10 to an expanded form can result in longitudinal or axial lengthening. View A illustrates the unexpanded auxetic stent 10, which has a proximal end demarcated by line 22, and a distal end demarcated by line 24. View B illustrates the stent following expansion, to create expanded auxetic stent 15. Line 24 still demarcates the distal end of the expanded auxetic stent 15. However, the proximal end 20 of the expanded auxetic stent 15 is past the line 22, thus illustrating how the expanded auxetic stent 15 has increased in length longitudinally following deployment and expansion. In embodiments, this longitudinal or axial length increase can be accommodated by a delivery device that can alter its configuration as an auxetic stent or similar device axially or longitudinally lengthens in response to expansion. Ex vivo, the auxetic stent in FIG. 1 is 6 cm in longitudinal length in the crimped/loaded state such as View A and 7.7 cm in longitudinal length in the deployed state such as in View B. It is this lengthening of the stent 10 to the expanded auxetic stent 15 that is addressed by the disclosed delivery device discussed below, to allow the stent to be positioned and expanded in a controlled fashion to optimize its efficacy and minimize the possibility of harm to any surrounding structures or diminishing of the stent's effectiveness.

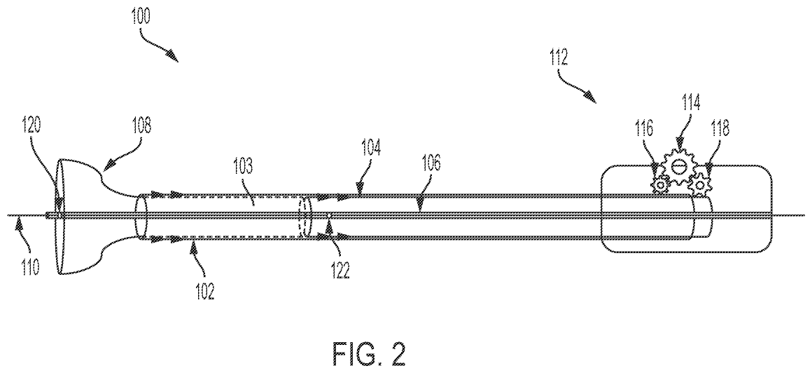

FIG. 2 illustrates the various components of an example over-the-wire delivery device 100, according to one possible embodiment. Device 100 includes an outer constraining device (or catheter) 102, into which a longitudinal controlling device 104 is inserted, thus housing the longitudinal controlling device 104 concentrically. Through the center of device 100 central core 106 runs longitudinally. The distal end of longitudinal controlling device 104 may be longitudinally offset from the distal end of the outer constraining device 102 to form a chamber 103, as can been seen. At the distal end and into this chamber 103 is inserted an auxetic device 108, which is a stent in the depicted embodiment. The auxetic device 108 is depicted in FIG. 2 as being partially deployed and partially expanded. Disposed within the central core 106 is a guidewire 110. The guidewire 110 may be part of a catheter or otherwise attached to a catheter or a similar structure, intended to guide the device 100 and inserted auxetic device, for delivery into a patient. The central core 106, similar to auxetic device 108, may be hollow, to accept the guidewire 110 for insertion into the patient.

The guidewire 110, in the depicted embodiment, may include a distal stent location radiopaque marker 120 and a final proximal stent location radiopaque marker 122 to facilitate visualizing placement of the device 100 and inserted auxetic device 108 within a patient, such as with an appropriate imaging device, e.g. CT scanner, X-ray imager, etc. The markers 120 and 122 may be positioned along the guidewire 110 at positions that correspond to the distal location of the auxetic device 108, as well as the anticipated final location and/or final length of the deployed auxetic device 108, accounting for its expected elongation. The guidewire 110 may be fabricated from any suitable material that is compatible with the particular procedure for which device 100 is employed. The guidewire 110 may be fabricated from a bio-compatible material that also can accept a radiopaque marker; in such an embodiment, guidewire 110 may be fabricated from a material that is otherwise transparent to whatever imaging device is employed to determine position of the device 100, to allow the radiopaque markers 120 and 122 to be positively identified. In other embodiments, guidewire 110 may be omitted, such as where the outer constraining device 102 and/or longitudinal controlling device 104 provide sufficient rigidity and/or allow for proper imaging of the device 100 to ascertain its position within a patient. In such embodiments, outer constraining device 102 and/or longitudinal controlling device 104 may be equipped with radiopaque markers.

The auxetic device 108, when loaded in the chamber 103 about central core 106, is restrained by the outer constraining device 102, and abuts or nearly abuts a distal end of the longitudinal controlling device 104, in the depicted embodiment. Between the central core 106 and outer constraining device 102, longitudinal controlling device 104 is configured to control the axial/longitudinal length of the auxetic device 108 during deployment. In other embodiments, as will be discussed herein, the auxetic device 108 may be inserted over a portion of the longitudinal controlling device 104, with the longitudinal controlling device 104 extending nearly to the open end of the outer constraining device 102, where the auxetic device 108 is deployed into a patient. In such an embodiment, chamber 103 may initially be minimal or non-existent, with chamber 103 potentially appearing and expanding as the auxetic device 108 is deployed, depending upon the configuration of the gear mechanism 112.

The outer constraining device 102, in embodiments, may comprise a catheter or similar tubular structure, sized to contain the auxetic device 108 in a configuration for deployment. In such a configuration, auxetic device 108 may be compressed to its smallest diameter, to minimize the diameter of the outer constraining device 102. When so sized, outer constraining device 102 also prevents expansion and/or controls the size of the auxetic device 108 until deployment, at which point the diameter of the auxetic device 108 may be expanded to the size required by the procedure being performed. The longitudinal controlling device 104, as can be seen in the depicted embodiment, is sized to a smaller diameter than the inner diameter of the outer constraining device 102. In this size, the longitudinal controlling device 104 is capable of sliding axially along the outer constraining device 102. When the longitudinal controlling device 104 slides relative to the outer constraining device 102 such that chamber 103 reduces in size, longitudinal controlling device 104 can act as a pusher or stop for deploying the auxetic device 108. The outer constraining device 102 is thus allowed to retract from the auxetic device 108, so that it is deployed into the surrounding anatomical structure. This motion may be accomplished by the configuration of the gear mechanism 112, as will be discussed further herein. In other embodiments, longitudinal controlling device 104 may extend partially or fully into the center of the auxetic device 108, which may be hollow, as typical of a stent, where an end of the longitudinal controlling device 104 is tapered or otherwise sized smaller than an inner diameter of the auxetic device 108. In still other embodiments, the portion of the longitudinal controlling device 104 that extends into auxetic device 108 may be configured to expand, such as via a balloon, to further allow control of expansion and shaping of the auxetic device 108 during deployment.

As the auxetic device 108 is deployed, it may either automatically expand and lengthen to fill the anatomical structure into which it is placed, and/or may be sized by the device 100, such as at least partially by action of the longitudinal controlling device 104. In implementations where auxetic device 108 automatically expands and lengthens to its proper deployed shape, such expansion may be effected by the nature of the construction of auxetic device 108, e.g. auxetic device 108 may be fabricated from a memory-type material that reverts to a predetermined shape upon exposure to ambient body heat. Such an example is visible in FIG. 2, where the end of auxetic stent 108 that has emerged from the outer constraining device 102 is depicted as having expanded to a significantly larger diameter over the end of outer constraining device 102.

As seen in the example embodiment of FIG. 2, the outer constraining device 102 is coupled to the longitudinal controlling device 104 via a gear mechanism 112 to allow active control of the axial/longitudinal length of the auxetic device 108. Gear mechanism 112, in the depicted embodiment, is comprised of a user manipulated or user controlled mechanism 114, implemented as a geared thumbwheel or finger wheel in the depicted embodiment. Gear mechanism 112 is disposed at an end of device 100 that is opposite and distal to the end of device 100 including chamber 103 and containing the auxetic device 108. This distal end is typically outside of the patient, and positioned so that the person performing or assisting in the procedure placing the auxetic device 108 may manipulate the gear mechanism 112 to effect deployment of the auxetic device 108. While the user controlled mechanism 114 in the depicted embodiment is a thumbwheel, user controlled mechanism 114 may be any suitable control that allows for the actuation of gear mechanism 112. In some other embodiments, user controlled mechanism 114 may be one or more levers, dials, wheels, sliders, buttons, or another suitable control. In some embodiments, user controlled mechanism 114 may be an interface to a motor drive, which is responsible for powering the gear mechanism 112 or directly manipulating the outer constraining device 102 and/or longitudinal controlling device 114.

Gear mechanism 112 may include one or more gears, depending upon the requirements of a given implementation. In the depicted embodiment, the user controlled mechanism 114 is mechanically coupled to a first gear 116 and second gear 118. The first gear 116 is in turn coupled to the outer constraining device 102, while the second gear 118 is coupled to the longitudinal controlling device 104. This coupling could be done by use of one or a multiple of any type of known gear including, but not limited to, compound gears, spiral bevel gears, rack and pinion gears, internal gears, worm gears, herringbone gears, helical gears, miter gears, screw gears, and/or beveled gears. Rotation or actuation of the user controlled mechanism 114, which, as discussed above, may be a thumbwheel or other user-manipulated device, in turn causes the first gear 116 and second gear 118 to rotate. Other embodiments may utilize a gear mechanism 112 with fewer or more gears, and may use a combination of one or more different types of gears. The selection of number, type, and/or size of gears may be made to achieve a specific movement of the outer constraining device 102 relative to the longitudinal controlling device 104, as may be required by a given embodiment or implementation.

In still other embodiments, gear mechanism 112 may be implemented without gears or with minimal gearing, such as via one or more actuators that directly act on the outer constraining device 102 and/or longitudinal controlling device 104. For example, mechanism 112 may be configured to be powered, with user controlled mechanism 114 comprising one or more buttons or toggles that activate one or more actuators. The actuators, in turn, can control movement of the constraining device 102 and/or longitudinal controlling device 104. The actuators may be configured to cause the constraining device 102 to move at a different rate than longitudinal controlling device 104, so that the auxetic device 108 is deployed in a controlled fashion from the end of constraining device 102. Gear mechanism 112 may be contained within a suitable housing that facilitates manipulation by a person assisting in placement of the auxetic device 108.

As may be seen in the example embodiment of FIG. 2, the first gear 116 may be of a different size than second gear 118. In such an embodiment, the first gear 116 rotates at a different rotation rate/frequency from second gear 118 when the user controlled mechanism 114 is rotated. Rotation of the user controlled mechanism 114, in some embodiments, may thus cause the outer constraining device 102 and longitudinal controlling device 104 to both translate along the guidewire 110/central core 106 in the same direction, but at different linear translation rates, through a mechanism such as, but not limited to, a rack and pinion gear mechanism. As a result, the longitudinal controlling device 104 will slide axially within the outer constraining device 102, causing the chamber 103 formed at the distal end between the outer constraining device 102 and the longitudinal controlling device 104 to axially lengthen or shorten, depending upon the direction that the user controlled mechanism 114 is rotated and the configuration of first gear 116 to second gear 118.

In embodiments, the ratio of the first gear 116 to the second gear 118 may be such that the outer constraining device 102 retracts at approximately the same rate as the longitudinal controlling device 104. In such embodiments, the chamber 103 may remain relatively static in size. I n other embodiments, the ratio of the first gear 116 to the second gear 118 may be such that the outer constraining device 102 may retract at a slower rate than the longitudinal controlling device 104, such as where the auxetic device 108 is configured to substantially lengthen as it is deployed, necessitating chamber 103 to expand in size to accommodate the lengthening in a controlled fashion. In still other embodiments, the ratio of the first gear 116 to the second gear 118 may be such that the outer constraining device 102 may retract at a faster rate than longitudinal controlling device 104. In such embodiments, chamber 103 may decrease in size, with longitudinal controlling device 104 acting to positively push the auxetic device 108 out of the end of the outer constraining device 102. As a variant, in some embodiments only outer constraining device 102 may retract, with longitudinal controlling device 104 essentially remaining static.

It will be recognized that the degree to which the chamber 103 axially lengthens or shortens, defined as the relative movement of the outer constraining device 102 against the longitudinal controlling device 104, will depend upon the size ratio between the first gear 116 and the second gear 118. This ratio may further be established by incorporation of additional gear mechanisms such as one or a multiple of any type of known gear including, but not limited to, compound gears, spiral bevel gears, rack and pinion gears, internal gears, worm gears, herringbone gears, helical gears, miter gears, screw gears, and/or beveled gears. The rotation rate/frequency of the first gear 116 and second gear 118 can be further relatively adjusted by incorporation of additional gear mechanisms such as one or a multiple of any type of known gear including, but not limited to, compound gears, spiral bevel gears, rack and pinion gears, internal gears, worm gears, herringbone gears, helical gears, miter gears, screw gears, and/or beveled gears. It should be appreciated that first gear 116 and second gear 118 need not actually be single gears, but rather represent control mechanisms tied to the outer constraining device 102, for first gear 116, and longitudinal controlling device 104, for second gear 118. The relative movement of the outer constraining device 102 to longitudinal controlling device 104 may be effected by any suitable mechanism.

Because the auxetic device 108 abuts the longitudinal controlling device 104, the auxetic device 108 is propelled from the device 100 at least partially in response to actuation of the user controlled mechanism 114. As the auxetic device 108 leaves the device 100, it may radially expand either due to spring tension inherent in the auxetic device 108 (such as resulting from materials of the auxetic device 108 reacting to body heat), or due to mechanical expansion, such as from a catheter balloon. Owing to its auxetic nature, as the auxetic device 108 radially expands it likewise axially lengthens. The change in axial dimension of the chamber due to differing linear translation rates of outer constraining device 102 and longitudinal controlling device 104 acts to accommodate this longitudinal lengthening, allowing for a controlled deployment of the auxetic device 108.

Other possible embodiments of device 100 may employ a passive mechanism instead of the gear mechanism 112. For example, a spring mechanism may be used to couple the outer constraining device 102 to the longitudinal controlling device 104, so that the chamber length changes automatically, e.g. without requiring direct manipulation of the outer constraining device 102 relative to the longitudinal controlling device 104, as the device 100 is used to deploy the auxetic device 108.

Delivery lengths for the auxetic stent/device delivery systems, including device 100, would include standard working lengths from 65 cm to 150 cm in some embodiments. However, embodiments that provide a short delivery system with a working length of 20-40 cm may be employed, such as for peripheral and/or intracranial applications. These shorter device embodiments may offer a unique workability for short vascular access to treatment site lengths. One specific use would be for retrograde access from the internal jugular vein to intracranial vein delivery site (such as transverse sinus). Another specific application would be retrograde access from common femoral/femoral vein to iliac vein delivery site.

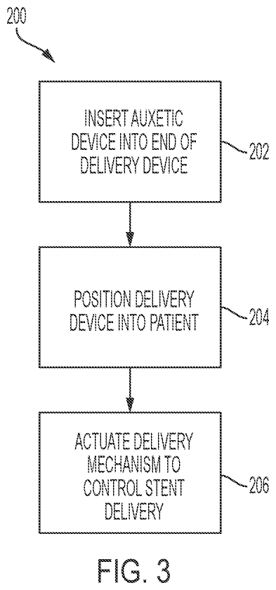

FIG. 3 illustrates the operations of an example method 200 for delivery of an auxetic device, such as a stent, using a delivery device, such as device 100. In operation 202, an auxetic device, such as a stent or auxetic device 108, may be inserted into an end of the delivery device, such as a chamber formed by concentric inner and outer devices, like chamber 103 formed by outer constraining device 102 and longitudinal controlling device 104. Depending upon the implementation, the auxetic device may be placed within the chamber but ahead of the inner device, or may be placed at least partially surrounding the inner device. The chamber itself is open, so that the auxetic device can be delivered into an appropriate site within a patient by action of the inner device relative to the outer device.

In operation 204, the end of the delivery device that is loaded with the auxetic device is inserted into the patient, and positioned into the appropriate position for delivery. Proper placement may be effected by reference to radiopaque markers that are positioned upon the end of the delivery device. These markers may be incorporated into a part of the delivery device, such as the outer device, the inner device, or a guidewire that may run through a core of the delivery device, with the inner and outer devices respectively positioned concentrically outward from the guidewire.

In operation 206, once the proper or desired position for the auxetic device is achieved, the delivery device is retracted and, depending upon the configuration of the delivery device, a delivery mechanism may be activated to cause the auxetic device to be expelled from the delivery device in a controlled fashion. The delivery mechanism may be a geared mechanism, such as gear mechanism 112, which is attached to the delivery device and actuated by an operator to cause the inner and outer devices to move relative to each other at predetermined different speeds. The auxetic device may radially expand or be expanded as it is delivered. The delivery device, with the inner and outer devices moving relative to each other from actuation of the geared mechanism, acts to control delivery of the auxetic device to accommodate its accompanying longitudinal expansion. Thus, by manipulation of the delivery mechanism in consideration of the auxetic properties of the auxetic device, the expansion and lengthening of the auxetic device can be relatively precisely controlled to optimize delivery.

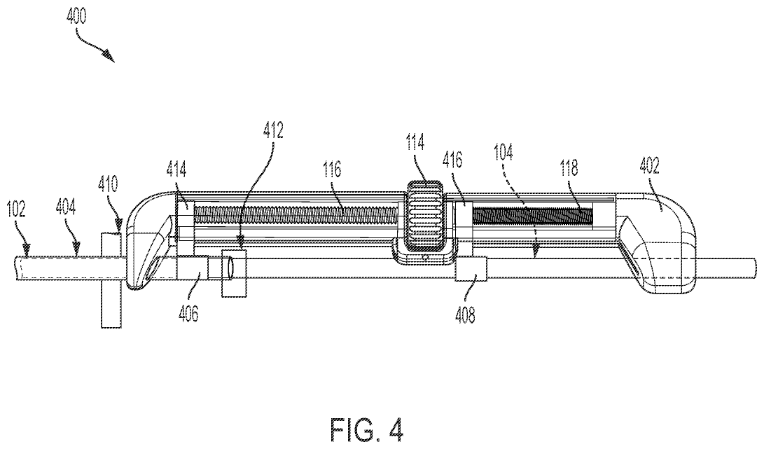

FIG. 4 illustrates an example delivery control mechanism 400 according to a possible embodiment, which may include or implement gear mechanism 112. Mechanism 400 includes a housing 402 which contains a user controlled mechanism 114, depicted as a thumb wheel, which is connected to first gear 116 and second gear 118 so that rotating the user controlled mechanism 114 imparts rotation to the first gear 116 and second gear 118. The outer constraining device 102, an outer sheath, and the inner longitudinal controlling device 104, an inner pusher, are coupled to the mechanism 400 and pass through the housing 402. The reader is referred to the description associated with FIG. 2, above, for greater detail on these structures. The outer constraining device 102 and inner longitudinal controlling device 104 engage with mechanism 400 by first passing through a groin sheath 404, and through two ferrules or apertures 410, for the outer sheath, and 412, for the inner pusher. The sheath 404 and apertures 410, 412, help the constraining device 102 and controlling device 404 move correctly though mechanism 400 when the user controlled mechanism 114 is actuated, and without binding.

In the depicted embodiment, the first gear 116 couples to the outer constraining device 102 via a runner 406, and the second gear 118 couples to the inner longitudinal controlling device 104 via a runner 408. Runner 406 is fixedly attached to the outer constraining device 102 such that axial movement of runner 406 will cause the outer constraining device 102 to likewise move axially. Similarly, runner 408 is fixedly attached to the inner longitudinal controlling device 104 such that axial movement of runner 408 will cause the inner longitudinal controlling device to move axially. Consequently, when either runner 406 or runner 408 moves relative to the other, the outer constraining device 102 and longitudinal controlling device 104 correspondingly move relative to each other, with the longitudinal controlling device 104 sliding axially within the outer constraining device 102.

In the depicted embodiment, runner 406 includes a collar 414 which is engaged to the first gear 116. The collar 414 includes an inner bore with threads that are complementary and of the same pitch as first gear 116. Likewise, runner 408 includes a collar 416 which is engaged to the second gear 118, which also includes an inner bore with threads that are complementary and of the same pitch as second gear 118. As can be seen in the depicted embodiment, first gear 116 has a coarser pitch than second gear 118. Rotation of the user controlled mechanism 114 causes both the first gear 116 and second gear 118 to rotate in synchronization. Due to the differing pitch, the coarser pitch of first gear 116 causes the runner 406 to travel down the first gear 116 towards the user controlled mechanism 114 at a greater rate than runner 408, which travels slower away from the user controlled mechanism 114 due to the finer pitch of second gear 118. Thus, as user controlled mechanism 114 is rotated, the outer constraining device 102 withdraws at a faster rate than the longitudinal controlling device 104 is withdrawn. As a result, an auxetic device placed within the end of the outer constraining device 102 is propelled out of the outer constraining device 102 by the longitudinal controlling device 104, which does not retreat as quickly, and, relative to outer constraining device 102, advances within the outer constraining device 102 towards its in that may be located within a patient.

It will be further observed that the first gear 116 is longer than the second gear 118 in the depicted embodiment. As a result, both runners 406 and 408 may reach the end of their respective gears at approximately the same time as the user controlled mechanism 114 is rotated. It will further be understood that the runners 406, 408 may advance or retreat depending upon the direction in which the user controlled mechanism 114 is rotated. First gear 116 and second gear 118, due to their implementation as worm gears or jack screws, can facilitate positive control of the constraining device 102 and controlling device 104 by resisting back feeding, viz. the worm gears resist turning when pressure is applied to either of the runners 406 or 408, helping to hold the devices 102 and 104 in place. While mechanism 400 implements first gear 116 and second gear 118 as worm gears, as discussed above, other embodiments may utilize different mechanisms for first gear 116 and/or second gear 118, such as conventional rotating gears, rack and pinion, or another arrangement. Further, as discussed above, the user controlled mechanism 114 can be implemented as a motor, which can cause the first gear 116 and second gear 118 to rotate upon electronic command. Still further, rather than first gear 116 and second gear 118, mechanism 400 could implement servos or actuators connected to runners 406 and/or 408, which are then electronically activated. First gear 116 and/or second gear 118 may be constructed from any suitable material, such as metal, plastic, composite, wood, ceramic, or another suitable material.

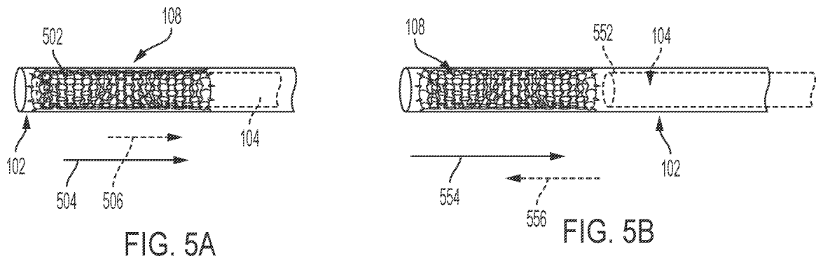

FIGS. 5A and 5B illustrate two possible arrangements of the auxetic device 108 when inserted into the device 100 for deployment into a patient. FIG. 5A illustrates auxetic device 108 inserted into the outer constraining device 102 and around the longitudinal controlling device 104, which is disposed concentrically within the auxetic device 108. As can be seen, the controlling device 104 has an end 502 that is tapered down and fit within the center channel of the auxetic device 108, so that it is near the end opening of both the auxetic device 108 and the constraining device 102. In this arrangement, when the constraining device 102 and controlling device 104 are retracted, the first and second gears may be configured so that both the constraining device 102 and controlling device 104 retract in the same direction, with constraining device 102 retracting at a greater rate 504 than the controlling device 104, at a lesser rate 506. This configuration may be particularly useful when the auxetic device 108 expands and lengthens at a rate equal to or greater than the speed at which the constraining device 102 retracts towards the end 502 of the controlling device 104.

FIG. 5B likewise illustrates auxetic device 108 inserted into outer constraining device 102, but abutting an end 552 of longitudinal controlling device 104, rather than concentrically around longitudinal controlling device 104. In this arrangement, when the constraining device 102 and controlling device 104 are retracted, the first and second gears may be configured so that the constraining device 102 retracts in a different direction 554 from controlling device 104, which advances in direction 556, but at a slower rate than constraining device 102 retracts to direction 554. This configuration may be useful when the auxetic device 108 does not expand at a rapid rate and/or the auxetic device 108 needs to be pushed out of the constraining device 102 for proper placement.

The devices described herein may be used in the deployment of medical stents in various medical applications, including stents currently known in the arts and marketed for use. In some embodiments, the stents in question comprise those taught in PCT Published Application Number PCT/US2020/013156 (WO 2020/146777A1, Al-Hakim et al., Oregon Health & Science University), published 16 Jul. 2020, the contents of which are incorporated herein in their entirety

Additional auxetic stents, tubular liners, tubular grafts, shunts, and intravascular implants that may be deployed using the devices described herein include those disclosed in US2011/0029063 (Ma et al., 3 Feb. 2011), US Pat. No. 6,613,079 (Wolinsky et al., 2 Sep. 2003), US2006/0129227A1 (Hengelmolen, 15 Jun. 2006), US2007/0213838 (Hengelmolen, 13 Sep. 2007), US 2018/0116834 A1 (Longo et al., 3 May 2018), US 2019/0060052 A1 (Harrison et al., 28 Feb. 2019), and US 2019/0076276 A1 (Longo et al., 14 Mar. 2019).

It will further be appreciated that stents, particularly auxetic stents as described or referenced herein, may be deployed using the presently described devices in anatomical structures morphologically similar to, but distinct from, vessels such as veins or arteries to facilitate patency of said anatomical structures. Thus, various disclosed embodiments provide a more general method of treating a stricture in a lumen in a mammal, the method comprising implanting a stent as described herein into a lumen, duct, or canal in need thereof. As used herein, the term “stricture” refers to an abnormal narrowing or constriction of a canal, duct, or other lumen in the body that affects normal passage of material (blood, air, food, feces, lymph, urine, saliva, bile, etc.) through the canal, duct, or lumen. As used herein, the term “implanting” refers to placement of a stent as described herein into a position in a duct, canal, or lumen experiencing a stricture, and expanding the stent to treat or alleviate the stricture.

Exemplary non-venous and non-arterial stent deployment applications that may be accomplished with the devices described herein include implantation into the bile duct, urogenital tract, gastrointestinal tract, tracheobronchial structures, sinus tract, salivary glands, salivary tubules, salivary ducts, and lymphatic channels. Additional applications include use in surgical procedures such as surgical enterosteotomies, surgical arteriovenous fistulas and grafts, and surgical anastomosis of any two structures within the body.

Stents identical or similar to stents as described in the references above (or another embodiment herein) used in treating strictures in a cystic duct or common bile duct or in treating biliary tract diseases may be from about 1 mm to about 30 mm in diameter and from about 5 mm to about 200 mm in length, fully expanded (the specific stent measurements for each specific use herein, unless otherwise specifically stated, are at full expansion of the stent—all diameters are outside diameters). In some embodiments such stents for bile duct use may be from about 5 mm to about 15 mm in diameter and from about 20 mm to about 120 mm in length, fully expanded. In some embodiments, the bile duct stents may be from about 5 mm to about 10 mm in diameter and from about 20 mm to about 120 mm in length. Specific stents for use in biliary treatments include (diameter×length) 5 mm×20 mm, 5 mm×30 mm, 5 mm×40 mm, 5 mm×50 mm, 5 mm×mm, 5 mm×70 mm, 5 mm×80 mm, 5 mm×90 mm, 5 mm×100 mm, 5 mm×110 mm, 5 mm×120 mm, 5 mm×130 mm, 5 mm×140 mm, 5 mm×150 mm, 6 mm×20 mm, 6 mm×mm, 6 mm×40 mm, 6 mm×50 mm, 6 mm×60 mm, 6 mm×70 mm, 6 mm×80 mm, 6 mm×90 mm, 6 mm×100 mm, 6 mm×110 mm, 6 mm×120 mm, 6 mm×130 mm, 6 mm×140 mm, 6 mm×150 mm, 8 mm×20 mm, 8 mm×30 mm, 8 mm×40 mm, 8 mm×50 mm, 8 mm×60 mm, 8 mm×70 mm, 8 mm×80 mm, 8 mm×90 mm, 8 mm×100 mm, 8 mm×110 mm, 8 mm×120 mm, 8 mm×130 mm, 8 mm×140 mm, 8 mm×150 mm, 10 mm×20 mm, mm×30 mm, 10 mm×40 mm, 10 mm×50 mm, 10 mm×60 mm, 10 mm×70 mm, 10 mm×80 mm, 10 mm×90 mm, 10 mm×100 mm, 10 mm×110 mm, 10 mm×120 mm, 10 mm×130 mm, 10 mm×140 mm, and 10 mm×150 mm.

Stents identical or similar to stents as described or referenced herein (or another embodiment herein) used in treating strictures in the human ureter or in treating urinary tract diseases associated with a uretal stricture may be from about 1 mm to about 100 mm in diameter and from about 5 mm to about 500 mm in length (fully expanded). In other embodiments, uretal stents may comprise from about 1 mm to about 15 mm in diameter and a length of from about 5 mm to about 500 mm in length. In other embodiments, uretal stents may comprise from about 1 mm to about 12 mm in diameter and a length of from about 5 mm to about 500 mm in length. In further embodiments, uretal stents may comprise from about 1 mm to about 3 mm in diameter and a length of from about 5 mm to about 500 mm in length. In further embodiments, uretal stents may comprise from about 1 mm to about 2 mm in diameter and a length of from about 5 mm to about 500 mm in length. Specific stents of the design herein for ureter implantation include those having the diameter x length of 1 mm×10 mm, 1 mm×20 mm, 1 mm×40 mm, 1 mm×60 mm, 1 mm×80 mm, 1 mm×100 mm, 1 mm×120 mm, 1 mm×150 mm, 1 mm×200 mm, 1 mm×250 mm, 1 mm×300 mm, 1 mm×350 mm, 1 mm×400 mm, 1 mm×500 mm, 2 mm×10 mm, 2 mm×20 mm, 2 mm×40 mm, 2 mm×60 mm, 2 mm×80 mm, 2 mm×100 mm, 2 mm×120 mm, 2 mm×150 mm, 2 mm×200 mm, 2 mm×250 mm, 2 mm×300 mm, 2 mm×350 mm, 2 mm×400 mm, and 2 mm×500 mm.

Stents identical or similar to stents as described or referenced herein (or another embodiment herein) used in treating strictures in the gastrointestinal tract may be from about 1 mm to about 100 mm in diameter and from about 5 mm to about 500 mm in length (fully expanded).

Colonic stents identical or similar to stent 100 (or another embodiment herein) may be from about 20 mm to about 40 mm in diameter and from about 20 mm to about 150 mm in length. In some embodiments, the colonic stents may be from about 20 mm to about 35mm in diameter and from about 40 mm to about 140 mm in length. In other embodiments, the colonic stents may be from about 26 mm to about 32 mm in diameter and from about 40 mm to about 120 mm in length.

Esophageal stents identical or similar to stents as described or referenced herein (or another embodiment herein) may be from about 10 mm to about 25 mm in diameter and from about 3 cm to about 20 cm in length. In some embodiments, the colonic stents may be from about 15 mm to about 25 mm in diameter and from about 5 cm to about 15 cm in length. In other embodiments, the colonic stents may be from about 17 mm to about 23 mm in diameter and from about 5 cm to about 15 cm in length.

In some embodiments, the tracheobronchial stents may be from about 5 mm to about 25 mm in diameter and from about 10 mm to about 100 mm in length. In some embodiments the tracheobronchial stents may be from about 6 mm to about 22 mm in diameter and from about 10 mm to about 100 mm in length. Specific examples of tracheobronchial stent sizes for uses here include the expanded diameter×length combinations of from about 8 mm× about 20 mm, about 8 mm× about 30 mm, about 8 mm× about 40 mm, 10 mm× about 20 mm, about 10 mm× about 30 mm, about 10 mm× about 40 mm, about 10 mm× about 60 mm, 12 mm× about 20 mm, about 12 mm× about mm, about 12 mm× about 40 mm, about 12 mm× about 60 mm, 12 mm× about 80 mm, 14 mm× about 20 mm, about 14 mm× about 30 mm, about 14 mm× about 40 mm, about 14 mm× about 60 mm, 14 mm× about 80 mm, 16 mm× about 20 mm, about 16 mm×about 30 mm, about 16 mm× about 40 mm, about 16 mm× about 60 mm, 16 mm× about mm, 18 mm× about 20 mm, about 18 mm× about 30 mm, about 18 mm× about 40 mm, about 18 mm× about 60 mm, 18 mm× about 80 mm, 20 mm× about 20 mm, about 20 mm×about 30 mm, about 20 mm× about 40 mm, about 20 mm× about 60 mm, and about 20 mm× about 80 mm.

Examples of salivary duct stents of use herein include those from about 0.5 mm to about 3 mm in diameter and from about 1 mm to about 40 mm in length.

It will be apparent to those skilled in the art that various modifications and variations can be made in the disclosed embodiments of the disclosed device and associated methods without departing from the spirit or scope of the disclosure. Thus, it is intended that the present disclosure covers the modifications and variations of the embodiments disclosed above provided that the modifications and variations come within the scope of any claims and their equivalents.

The following are additional example embodiments:

Example 1 is a delivery device for an auxetic device, comprising a hollow tubular constraining device with a longitudinal axis; a longitudinal controlling device, sized to fit concentrically within the constraining device along its longitudinal axis; and a mechanism coupled to the constraining device and the controlling device, wherein an end of the controlling device is offset along the longitudinal axis of the constraining device from an end of the constraining device so as to form a chamber between the end of the constraining device and the end of the controlling device, the chamber configured to accept the auxetic device, and the mechanism is configured to cause a size of the chamber to be adjusted along the longitudinal axis of the constraining device.

Example 2 includes the subject matter of example 1, or some other example herein, further comprising a central core, the central core comprised of a first radiopaque marker disposed upon a distal end, and a second radiopaque marker disposed away from the distal end along the longitudinal axis of the constraining device at a position on the central core corresponding to a final location of the auxetic device.

Example 3 includes the subject matter of example 1 or 2, or some other example herein, wherein the mechanism is a user-actuated mechanism comprised of a plurality of gears coupled to the constraining device and the controlling device, and the chamber size is adjusted in response to a user actuating the mechanism.

Example 4 includes the subject matter of any of examples 1-3, or some other example herein, wherein each of the plurality of gears comprise a jack screw, and the constraining device and controlling device are each coupled to a respective one of the jack screws by a runner.

Example 5 includes the subject matter of example 4, or some other example herein, wherein the jack screw coupled to the constraining device has a different thread pitch than the jack screw coupled to the controlling device.

Example 6 includes the subject matter of example 5, or some other example herein, wherein the jack screw coupled to the constraining device has a coarser thread pitch than the jack screw coupled to the controlling device.

Example 7 includes the subject matter of any of examples 4-6, or some other example herein, wherein each of the jack screws is coupled to a thumb wheel such that rotation of the thumb wheel imparts a corresponding rotation to each of the jack screws.

Example 8 includes the subject matter of any of examples 4-7, or some other example herein, wherein each of the jack screws is coupled to a motor.

Example 9 includes the subject matter of example 1 or 2, or some other example herein, wherein the mechanism is a spring coupled to the constraining device and the controlling device.

Example 10 includes the subject matter of any of examples 1-9, or some other example herein, wherein the controlling device inserts at least partially into the auxetic device when the auxetic device is inserted into the chamber.

Example 11 includes the subject matter of any of examples 1-9, or some other example herein, wherein an end of the controlling device abuts an end of the auxetic device when the auxetic device is inserted into the chamber.

Example 12 includes the subject matter of any of examples 1-11, or some other example herein, wherein the auxetic device is a stent.

Example 13 is a method, comprising inserting an auxetic device into a chamber disposed at the end of a delivery device; positioning the delivery device into a tubular structure within a patient; and actuating a mechanism on the delivery device to place the auxetic device into the tubular structure; wherein the chamber is defined by a controlling structure and a tubular constraining structure disposed concentrically about the controlling structure, the controlling structure offset from an end of the constraining structure to form the chamber, and the mechanism is configured to cause a size of the chamber to be adjusted along the longitudinal axis of the constraining device.

Example 14 includes the subject matter of example 13, or some other example herein, wherein the mechanism comprises a thumbwheel, and actuating the mechanism comprises rotating the thumbwheel to cause the size of the chamber to decrease, forcing the auxetic device out of the chamber.

Example 15 includes the subject matter of example 13 or 14, or some other example herein, wherein actuating the mechanism causes the controlling structure and the constraining structure to move in a same direction, the controlling structure moving at a different speed than the constraining structure.

Example 16 includes the subject matter of example 13 or 14, or some other example herein, wherein actuating the mechanism causes the controlling structure and the constraining structure to move in opposite directions.

Example 17 includes the subject matter of any of examples 13-16, or some other example herein, wherein inserting the auxetic device into the chamber comprises abutting an end of the auxetic device against an end of the controlling structure.

Example 18 includes the subject matter of any of examples 13-16, or some other example herein, method of claim 13, wherein inserting the auxetic device into the chamber comprises disposing a portion of the auxetic device concentrically around an end of the controlling structure.

What is claimed is:

1. A delivery device for an auxetic device, comprising:
a hollow tubular constraining device with a longitudinal axis;
a longitudinal controlling device, sized to fit concentrically within the constraining device along its longitudinal axis; and
a mechanism coupled to the constraining device and the controlling device,
wherein:
an end of the controlling device is offset along the longitudinal axis of the constraining device from an end of the constraining device so as to form a chamber between the end of the constraining device and the end of the controlling device, the chamber configured to accept the auxetic device, and
the mechanism is configured to cause a size of the chamber to be adjusted along the longitudinal axis of the constraining device.

2. The delivery device of claim 1, further comprising a central core, the central core comprised of a first radiopaque marker disposed upon a distal end, and a second radiopaque marker disposed away from the distal end along the longitudinal axis of the constraining device at a position on the central core corresponding to a final location of the auxetic device.

3. The delivery device of claim 1, wherein the mechanism is a user-actuated mechanism comprised of a plurality of gears coupled to the constraining device and the controlling device, and the chamber size is adjusted in response to a user actuating the mechanism.

4. The delivery device of claim 3, wherein each of the plurality of gears comprise a jack screw, and the constraining device and controlling device are each coupled to a respective one of the jack screws by a runner.

5. The delivery device of claim 4, wherein the jack screw coupled to the constraining device has a different thread pitch than the jack screw coupled to the controlling device.

6. The delivery device of claim 5, wherein the jack screw coupled to the constraining device has a coarser thread pitch than the jack screw coupled to the controlling device.

7. The delivery device of claim 4, wherein each of the jack screws is coupled to a thumb wheel such that rotation of the thumb wheel imparts a corresponding rotation to each of the jack screws.

8. The delivery device of claim 4, wherein each of the jack screws is coupled to a motor.

9. The delivery device of claim 1, wherein the mechanism is a spring coupled to the constraining device and the controlling device.

10. The delivery device of claim 1, wherein the controlling device inserts at least partially into the auxetic device when the auxetic device is inserted into the chamber.

11. The delivery device of claim 1, wherein an end of the controlling device abuts an end of the auxetic device when the auxetic device is inserted into the chamber.

12. The delivery device of claim 1, wherein the auxetic device is a stent.

13. A method, comprising:
inserting an auxetic device into a chamber disposed at the end of a delivery device;
positioning the delivery device into a tubular structure within a patient; and
actuating a mechanism on the delivery device to place the auxetic device into the tubular structure;
wherein:
the chamber is defined by a controlling structure and a tubular constraining structure disposed concentrically about the controlling structure, the controlling structure offset from an end of the constraining structure to form the chamber, and
the mechanism is configured to cause a size of the chamber to be adjusted along the longitudinal axis of the constraining device.

14. The method of claim 13, wherein the mechanism comprises a thumbwheel, and actuating the mechanism comprises rotating the thumbwheel to cause the size of the chamber to decrease, forcing the auxetic device out of the chamber.

15. The method of claim 13, wherein actuating the mechanism causes the controlling structure and the constraining structure to move in a same direction, the controlling structure moving at a different speed than the constraining structure.

16. The method of claim 13, wherein actuating the mechanism causes the controlling structure and the constraining structure to move in opposite directions.

17. The method of claim 13, wherein inserting the auxetic device into the chamber comprises abutting an end of the auxetic device against an end of the controlling structure.

18. The method of claim 13, wherein inserting the auxetic device into the chamber comprises disposing a portion of the auxetic device concentrically around an end of the controlling structure.
